(19) United States
(12) Patent Application Publication
(10) Pub. No.: US 2006/0211600 A1
(43) Pub. Date: Sep. 21, 2006

(54) Stem cell-derived factors for treating pathologic conditions

(76) Inventors: Victor Dzau, Durham, NC (US); Maria Mirotsou, Dresden (DE)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/350,978

(22) Filed: Feb. 8, 2006

Related U.S. Application Data
(60) Provisional application No. 60/651,159, filed on Feb. 8, 2005.

(51) Int. Cl. A61K 38/17

(52) U.S. Cl. 514/2

(57) ABSTRACT

Compositions and methods for cellular and tissue protection, repair, and regeneration are described. Mesenchymal cell-derived paracrine factors confer a therapeutic benefit to a variety of injured, compromised or diseased tissues such as myocardial tissue.

### RELATED APPLICATIONS

This application claims U.S. Ser. No. 60/651,159, filed Feb. 8, 2005, which is incorporated herein by reference in its entirety.

### STATEMENT AS TO FEDERALLY SPONSORED RESEARCH

This invention was made with U.S. government support under National Institutes of Health grants. The government has certain rights in the invention.

### FIELD OF THE INVENTION

The invention relates to inhibiting cell damage.

### BACKGROUND OF THE INVENTION

Patient mortality and morbidity is increased by cell/tissue damage or death resulting from acute and chronic injury or disease of the heart muscle, such as myocardial infarction, cardiac failure, stroke, degenerative neurological disease, spinal injury, musculoskeletal diseases, hypertension, and diabetes. It is of great importance to determine methods and composition to prevent, reduce, and/or repair this damage.

### SUMMARY OF THE INVENTION

The invention is based upon the surprising discovery that paracrine factors secreted from mesenchymal stem cells (MSC), e.g., genetically modified bone marrow derived mesenchymal cells alone (i.e., in the absence of whole viable stem cells) confer a therapeutic benefit to bodily tissues. Thus, stem cells serve as a factory of biologic products that are purified and administered to subjects.

The paracrine factors are useful in cellular and tissue protection, repair, and regeneration. Mesenchymal stem cells or progenitor comprise an Akt gene (Akt-MSC). One or more secreted compounds (e.g., and isolated compound or a mixture of secreted compounds such as a MSC culture supernatant) confers a clinical benefit to a variety of injured, compromised, or disease tissues.

Accordingly, the invention features methods of inhibiting cell damage or inducing cell repair or regeneration by contacting the cell or tissue with one or more paracrine factors secreted by the Akt-MSCs. For example, the cells or tissues are contacted with the cell culture supernatant of cultured Akt-MSCs. Optionally, supernatant is fractionated to isolate one or more paracrine factor to produce a cytoprotective compound.

Factors derived from Akt-MSCs confer a therapeutic benefit at each stage of a hypoxic cardiac event (early, middle, and late stage). Early one, factors confer a cell protective effect, followed by inotropy, angiogenesis, and cardiac remodeling.

The invention also features methods of inhibiting cell damage, inducing cell repair or regeneration or inhibiting an ischemic or reperfusion related injury in a subject. Cell damage or injury is inhibited by administering to the subject or contacting a cell with a composition containing a purified cytoprotective compound such as a substantially pure polypeptide, or a mixture of substantially pure polypeptides. Similarly, cell repair or regeneration is induced by administering to the subject or contacting a cell with a composition containing a purified cytoprotective compound. Polypeptides or other compounds described herein are said to be “substantially pure” when they are within preparations that are at least 60% by weight (dry weight) the compound of interest. Preferably, the preparation is at least 75%, more preferably at least 90%, and most preferably at least 99%, by weight the compound of interest. Purity is measured by any appropriate standard method, for example, by column chromatography, polyacrylaminde gel electrophoresis, or HPLC analysis.

The cell is a cardiac cell such as a cardiomyocyte, a liver cell, a kidney cell, a liver cell, a neurological (e.g., brain, spinal cord) cell, or a pancreatic cell. Cell or tissue damage is defined by a loss or diminution of cell function. Such loss or decrease in function leads to eventual cell death. For example, a loss of cardiomyocyte function results in the loss of the contractile function of the cell. Cardiomyocytes that have lost their ability to contract form round cells rather that rod shaped cells when cultured. Ischemia causes irreversible cellular/tissue damage and cell death. Reperfusion exacerbates ischemic damage by activating inflammatory response and oxidative stress. Oxidative stress modifies membrane lipids, proteins and nucleic acids resulting in cellular/tissue damage or death, and depression of cardiac, endothelial and kidney function.

Also included in the invention are methods of regenerating an injured myocardial tissue by administered to the tissue a composition containing a cytoprotective compound. The cardiac muscle has been damaged by disease, such as a myocardial infarction. By regenerating an injured myocardial tissue is meant restoring ventricular function. Ventricular function is measured by methods known in the art such as radionuclide angiography.

A cytoprotective compound is a compound which is capable of inhibiting cell damage such as oxidative-stress induced cell death or apoptosis. Suitable cytoprotective compound include for example adipsin, adrenomedullin, chemokine (C—C motif) ligand 2, cysteine rich protein 61, lysyl oxidase-like 2, secreted frizzled-related sequence protein 2, or serine proteinase inhibitor.

The composition is administered to the subject prior to, at the time of, or shortly after (5, 10, 15, 30, 60 minutes; 1.5, 2, 4, 6, 12, 18, 24, 48 hours) identification of cell damage or identification of a symptom of ischemia or reperfusion injury. For example the composition is administered prior to a cardiac event. Symptoms include for example, chest pain, arm pain, fatigue and shortness of breath. For example, the composition is administered after a cardiac event such as a myocardial infarction. The composition is administered systemically or locally. For example, the composition is administered directly, i.e., by myocardial injection to the cardiac tissue. Optionally, the subject is further administered VEGF or thyrosin beta 4.

The composition is administered at a dose sufficient to inhibit apoptotic death or oxidative stress-induced cell death. To determine whether the composition inhibits oxidative-stress induced cell death, the composition is tested by incubating the composition with a primary or immortalized cell such as a cardiomyocyte. A state of oxidative stress of the cells is induced (e.g., by incubating them with H₂O₂), and cell viability is measured using standard methods. As a control, the cells are incubated in the absence of the composition and then a state of oxidative stress is induced. A decrease in cell death (or an increase in the number of viable cells) in the compound treated sample indicates that the composition inhibits oxidative-stress induced cell death. Alternatively, an increase in cell death (or an decrease in the number of viable cells) in the compound treated sample indicates that the composition does not inhibit oxidative-stress induced cell death. The test is repeated using different doses of the composition to determine the dose range in which the composition functions to inhibit oxidative-stress induced cell death.

A subject to be treated is suffering from or at risk of developing a condition characterized by aberrant cell damage such as oxidative-stress induced cell death (e.g., apoptotic cell death) or an ischemic or reperfusion related injury. A subject suffering from or at risk of developing such a condition is identified by the detection of a known risk factor, e.g., gender, age, high blood pressure, obesity, diabetes, prior history of smoking, stress, genetic or familial predisposition, attributed to the particular disorder, or previous cardiac event such as myocardial infarction or stroke.

Conditions characterized by aberrant cell damage or death include cardiac disorders (acute or chronic) such as stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, kidney ischemia, ischemic hepatitis, hepatic vein thrombosis, cirrhosis, portal vein thrombosis, pancreatitis, ischemic colitis, or myocardial hypertrophy.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a series of bar charts demonstrating ventricular function after MI and MSCs injection. Panel (a) LVSP 72 hours after MI was reduced in control PBS animals and was slightly but not significantly increased after injection of GFP-MSCs; in the Akt-MSCs group LVSP was significantly improved compared with both the PBS and GFP group. Panel (b) LV±dP/dt deteriorated both in the PBS and GFP group but not after Akt-MSCs injection. Panel(c) Improved LVSP values were also observed in the Akt group at 15 days after MI as compared with PBS and GFP groups. Panel(d) At 15 days −dP/dt was normalized in Akt-MSCs and +dP/dt was increased but not completely normalized. Statistics: * p<0.05 vs. sham; † p<0.05 vs. PBS, ‡ p<0.05 vs. GFP-MSCs.

FIG. 2 is a series of photographs showing the effect of MSCs transplantation on infarct size and inflammatory response. Panels (a-b) LCA ligation in PBS control group resulted in an infarct equivalent in size to a third of the entire left ventricular area as calculated by TTC staining and a massive infiltration of inflammatory cells as documented by H&E staining. Panels (c-d) After injection of GFP-MSCs infarct size and inflammatory response were reduced. Panels (e-f) Transplantation of Akt-MSCs dramatically limited infarct size as well as the inflammatory response.

FIG. 3 is a series of photographs showing Post-infarction ventricular remodeling at 2 weeks after infarction. Panels (a-d) Sham-operated animals show normal wall and chamber morphology. Panels (e-f) Marked thinning and scarring of the anterior wall and accompanying chamber enlargement are seen in the PBS-treated control animals. Panels (i-j) Injection of GFP-MSCs partially reduced anterior wall thinning and chamber dimension. Panels (m-n) Injection of Akt-MSCs markedly reduced collagen deposition and preserved wall and chamber dimensions.

FIG. 4A-D are a series of photomicrographs showing the effect of conditioned medium on Adult Rat Ventricular Cardiomyocytes (ARVCs) viability. Panel (a), considered as baseline; ARVCs after 24 hours of hypoxia in α-MEM; Panel (b), GFP-MSCs; Panel (c) or Akt-MSCs; Panel (d) N-M; and GFP-MSCs; Panel (e) or Akt-MSCs; Panel (f) H-M.

FIG. 4G is a bar graph summarizing the results (n=5 for each condition) of Figure A-D Statistics: * p<0.05 vs. control; † p<0.05 vs. GFP N-M; ‡ p<0.05 vs. GFP H-M; § p<0.05 vs. Akt N-M. Under all the condition tested the total and rod-shaped ARVCs were significantly fewer than at baseline.

FIG. 5A is a bar graph showing the effect of conditioned medium on apoptosis in ARVCs. The black bars represent results in presence of medium conditioned under normoxia; the white bars show caspase 3 activity of ARVCs cultured in medium conditioned under hypoxia. Statistics: * p<0.05 vs. normoxic and hypoxic CTR-M; † p<0.05 vs. normoxic GFP-M; ‡ p<0.05 vs. hypoxic GFP-M; § p<0.05 vs. normoxic Akt-M.

FIG. 5B-D are photographs showing the effect of conditioned medium on apoptosis in ARVCs. Representative pictures of total (red fluorescent) and TUNEL-positive (green fluorescent) ARVCs nuclei in presence of CTR-M (b-c), GFP-MSCs H-M (d-e) and Akt-MSCs H-M (f-g).

FIG. 6 is a series of bar graph showing post-myocardial infarction ex vivo cardiac function after injection of conditioned medium. Panel (a) LVSP 72 hours after the MI was reduced in animals injected with CTR-cM and GFP-cM; injection of Akt-cM resulted in improved LVSP. Panel (b), same results were obtained in terms of +dP/dt and the −dP/dt was normalized in hearts injected with Akt-cM. Panels (c-d) Akt-cM treated hearts exhibited significantly greater inotropic response to dobutamine than the other control groups. Statistics: * p<0.05 vs. sham; † p<0.05 vs. CTR-cM, ‡ p<0.05 vs. GFP-cM.

### DETAILED DESCRIPTION

The present invention is based upon the unexpected discovery of that MSC-secreted products confer a therapeutic benefit to injured or compromised tissues. Disclosed herein is a Akt-MSC mediated paracrine mechanism of organ protection and repair. More particularly, the invention provides purified cytoprotective polypeptides isolated from Akt-MSCs and methods of using these cytoprotective polypeptides to prevent myocardial damage and ventricular dysfunction.

Akt Genes

Akt-MSCs are produced by introducing (e.g., by retrovirus-mediated transduction) into mesenchymal stem cells isolated from the bone marrow an Akt coding sequence or fragment, e.g., Akt-1, Ak-2 or Akt-3. The Akt nucleic acid is human, mouse, or rat.

Exemplary human Akt-1 polypeptides include GenBank Accession numbers NP_{—}005154 and AAH00479. Exemplary human Akt-2 polypeptides includes for example GenBank Accession numbers P31751 and NP_{—}001617. Exemplary human Akt-3 polypeptides includes for example GenBank Accession numbers Q9Y243 and NP_{—}005456. Exemplary nucleic acids encoding Akt include human Akt-1 available at GENBANK™ Accession No. NM_{—}005163 (SEQ ID NO:1), human Akt-2 available at GENBANK™ Accession No. NM_{—}001626 (SEQ ID NO:2) and human Akt-3 available at GENBANK™ Accession No. AJ245709 (SEQ ID NO:3) (all of which are hereby incorporated by reference)or nucleic acids encoding the human Akt polypeptides described above. mRNA sequences and the corresponding coding region for human Akt are shown below.

| (SEQ ID NO:1) | |
|---|---|
| Akt-1 mRNA | |
| 1 | atcctgggac agggcacagg gccatctgtc accaggggct tagggaaggc cgagccagcc |
| 61 | tgggtcaaag aagtcaaagg ggctgcctgg aggaggcagc ctgtcagctg gtgcatcaga |
| 121 | ggctgtggcc aggccagctg ggctcgggga gcgccagcct gagaggagcg cgtgagcgtc |
| 181 | gcgggagcct cgggcaccat gagcgacgtg gctattgtga aggagggttg gctgcacaaa |
| 241 | cgaggggagt acatcaagac ctggcggcca cgctacttcc tcctcaagaa tgatggcacc |
| 301 | ttcattggct acaaggagcg gccgcaggat gtggaccaac gtgaggctcc cctcaacaac |
| 361 | ttctctgtgg cgcagtgcca gctgatgaag acggagcggc cccggcccaa caccttcatc |
| 421 | atccgctgcc tgcagtggac cactgtcatc gaacgcacct tccatgtgga gactcctgag |
| 481 | gagcgggagg agtggacaac cgccatccag actgtggctg acggcctcaa gaagcaggag |
| 541 | gaggaggaga tggacttccg gtcgggctca cccagtgaca actcaggggc tgaagagatg |
| 601 | gaggtgtccc tggccaagcc caagcaccgc gtgaccatga acgagtttga gtacctgaag |
| 661 | ctgctgggca agggcacttt cggcaaggtg atcctggtga aggagaaggc cacaggccgc |
| 721 | tactacgcca tgaagatcct caagaaggaa gtcatcgtgg ccaaggacga ggtggcccac |
| 781 | acactcaccg agaaccgcgt cctgcagaac tccaggcacc ccttcctcac agccctgaag |
| 841 | tactctttcc agacccacga ccgcctctgc tttgtcatgg agtacgccaa cgggggcgag |
| 901 | ctgttcttcc acctgtcccg ggaacgtgtg ttctccgagg accgggcccg cttctatggc |
| 961 | gctgagattg tgtcagccct ggactacctg cactcggaga agaacgtggt gtaccgggac |
| 1021 | ctcaagctgg agaacctcat gctggacaag gacgggcaca ttaagatcac agacttcggg |
| 1081 | ctgtgcaagg aggggatcaa ggacggtgcc accatgaaga ccttttgcgg cacacctgag |
| 1141 | tacctggccc ccgaggtgct ggaggacaat gactacggcc gtgcagtgga ctggtggggg |
| 1201 | ctgggcgtgg tcatgtacga gatgatgtgc ggtcgcctgc ccttctacaa ccaggaccat |
| 1261 | gagaagcttt ttgagctcat cctcatggag gagatccgct tcccgcgcac gcttggtccc |
| 1321 | gaggccaagt ccttgctttc agggctgctc aagaaggacc ccaagcagag gcttggcggg |
| 1381 | ggctccgagg acgccaagga gatcatgcag catcgcttct ttgccggtat cgtgtggcag |
| 1441 | cacgtgtacg agaagaagct cagcccaccc ttcaagcccc aggtcacgtc ggagactgac |
| 1501 | accaggtatt ttgatgagga gttcacggcc cagatgatca ccatcacacc acctgaccaa |
| 1561 | gatgacagca tggagtgtgt ggacagcgag cgcaggcccc acttccccca gttctcctac |
| 1621 | tcggccagca gcacggcctg aggcggcggt ggactgcgct ggacgatagc ttggagggat |
| 1681 | ggagaggcgg cctcgtgcca tgatctgtat ttaatggttt ttatttctcg ggtgcatttg |
| 1741 | agagaagcca cgctgtcctc tcgagcccag atggaaagac gtttttgtgc tgtgggcagc |
| 1801 | accctccccc gcagcggggt agggaagaaa actatcctgc gggttttaat ttatttcatc |
| 1861 | cagtttgttc tccgggtgtg gcctcagccc tcagaacaat ccgattcacg tagggaaatg |
| 1921 | ttaaggactt ctacagctat gcgcaatgtg gcattggggg gccgggcagg tcctgcccat |
| 1981 | gtgtcccctc actctgtcag ccagccgccc tgggctgtct gtcaccagct atctgtcatc |
| 2041 | tctctggggc cctgggcctc agttcaacct ggtggcacca gatgcaacct cactatggta |
| 2101 | tgctggccag caccctctcc tgggggtggc aggcacacag cagcccccca gcactaaggc |
| 2161 | cgtgtctctg aggacgtcat cggaggctgg gcccctggga tgggaccagg gatgggggat |
| 2221 | gggccagggt ttacccagtg ggacagagga gcaaggttta aatttgttat tgtgtattat |
| 2281 | gttgttcaaa tgcattttgg gggtttttaa tctttgtgac aggaaagccc tcccccttcc |
| 2341 | ccttctgtgt cacagttctt ggtgactgtc ccaccggagc ctccccctca gatgatctct |
| 2401 | ccacggtagc acttgacctt ttcgacgctt aacctttccg ctgtcgcccc aggccctccc |
| 2461 | tgactccctg tgggggtggc catccctggg cccctccacg cctcctggcc agacgctgcc |
| 2521 | gctgccgctg caccacggcg tttttttaca acattcaact ttagtatttt tactattata |
| 2581 | atataatatg gaaccttccc tccaaattct |
| Coding sequence = nucleotide 199-1641. | |
| (SEQ ID NO:2) | |
| Akt-2 mRNA | |
| 1 | gaattccagc ggcggcgccg ttgccgctgc cgggaaacac aaggaaaggg aaccagcgca |
| 61 | gcgtggcgat gggcgggggt agagccccgc cggagaggct gggcggctgc cggtgacaga |
| 121 | ctgtgccctg tccacggtgc ctcctgcatg tcctgctgcc ctgagctgtc ccgagctagg |
| 181 | tgacagcgta ccacgctgcc accatgaatg aggtgtctgt catcaaagaa ggctggctcc |
| 241 | acaagcgtgg tgaatacatc aagacctgga ggccacggta cttcctgctg aagagcgacg |
| 301 | gctccttcat tgggtacaag gagaggcccg aggcccctga tcagactcta ccccccttaa |
| 361 | acaacttctc cgtagcagaa tgccagctga tgaagaccga gaggccgcga cccaacacct |
| 421 | ttgtcatacg ctgcctgcag tggaccacag tcatcgagag gaccttccac gtggattctc |
| 481 | cagacgagag ggaggagtgg atgcgggcca tccagatggt cgccaacagc ctcaagcagc |
| 541 | gggccccagg cgaggacccc atggactaca agtgtggctc ccccagtgac tcctccacga |
| 601 | ctgaggagat ggaagtggcg gtcagcaagg cacgggctaa agtgaccatg aatgacttcg |
| 661 | actatctcaa actccttggc aagggaacct ttggcaaagt catcctggtg cgggagaagg |
| 721 | ccactggccg ctactacgcc atgaagatcc tgcgaaagga agtcatcatt gccaaggatg |
| 781 | aagtcgctca cacagtcacc gagagccggg tcctccagaa caccaggcac ccgttcctca |
| 841 | ctgcgctgaa gtatgccttc cagacccacg accgcctgtg ctttgtgatg gagtatgcca |
| 901 | acgggggtga gctgttcttc cacctgtccc gggagcgtgt cttcacagag gagcgggccc |
| 961 | ggttttatgg tgcagagatt gtctcggctc ttgagtactt gcactcgcgg gacgtggtat |
| 1021 | accgcgacat caagctggaa aacctcatgc tggacaaaga tggccacatc aagatcactg |
| 1081 | actttggcct ctgcaaagag ggcatcagtg acggggccac catgaaaacc ttctgtggga |
| 1141 | ccccggagta cctggcgcct gaggtgctgg aggacaatga ctatggccgg gccgtggact |
| 1201 | ggtgggggct gggtgtggtc atgtacgaga tgatgtgcgg ccgcctgccc ttctacaacc |
| 1261 | aggaccacga gcgcctcttc gagctcatcc tcatggaaga gatccgcttc ccgcgcacgc |
| 1321 | tcagccccga ggccaagtcc ctgcttgctg ggctgcttaa gaaggacccc aagcagaggc |
| 1381 | ttggtggggg gcccagcgat gccaaggagg tcatggagca caggttcttc ctcagcatca |
| 1441 | actggcagga cgtggtccag aagaagctcc tgccaccctt caaacctcag gtcacgtccg |
| 1501 | aggtcgacac aaggtacttc gatgatgaat ttaccgccca gtccatcaca atcacacccc |
| 1561 | ctgaccgcta tgacagcctg ggcttactgg agctggacca gcggacccac ttcccccagt |
| 1621 | tctcctactc ggccagcatc cgcgagtgag cagtctgccc acgcagagga cgcacgctcg |
| 1681 | ctgccatcac cgctgggtgg ttttttaccc ctgcc |
| Coding sequence = nucleotide 204-1649. | |
| (SEQ ID NO:3) | |
| Akt-3 mRNA | |
| 1 | gggagtcatc atgagcgatg ttaccattgt gaaagaaggt tgggttcaga agaggggaga |
| 61 | atatataaaa aactggaggc caagatactt ccttttgaag acagatggct cattcatagg |
| 121 | atataaagag aaacctcaag atgtggattt accttatccc ctcaacaact tttcagtggc |
| 181 | aaaatgccag ttaatgaaaa cagaacgacc aaagccaaac acatttataa tcagatgtct |
| 241 | ccagtggact actgttatag agagaacatt tcatgtagat actccagagg aaagggaaga |
| 301 | atggacagaa gctatccagg ctgtagcaga cagactgcag aggcaagaag aggagagaat |
| 361 | gaattgtagt ccaacttcac aaattgataa tataggagag gaagagatgg atgcctctac |
| 421 | aacccatcat aaaagaaaga caatgaatga ttttgactat ttgaaactac taggtaaagg |
| 481 | cacttttggg aaagttattt tggttcgaga gaaggcaagt ggaaaatact atgctatgaa |
| 541 | gattctgaag aaagaagtca ttattgcaaa ggatgaagtg gcacacactc taactgaaag |
| 601 | cagagtatta aagaacacta gacatccctt tttaacatcc ttgaaatatt ccttccagac |
| 661 | aaaagaccgt ttgtgttttg tgatggaata tgttaatggg ggcgagctgt ttttccattt |
| 721 | gtcgagagag cgggtgttct ctgaggaccg cacacgtttc tatggtgcag aaattgtctc |
| 781 | tgccttggac tatctacatt ccggaaagat tgtgtaccgt gatctcaagt tggagaatct |
| 841 | aatgctggac aaagatggcc acataaaaat tacagatttt ggactttgca aagaagggat |
| 901 | cacagatgca gccaccatga agacattctg tggcactcca gaatatctgg caccagaggt |
| 961 | gttagaagat aatgactatg gccgagcagt agactggtgg ggcctagggg ttgtcatgta |
| 1021 | tgaaatgatg tgtgggaggt tacctttcta caaccaggac catgagaaac tttttgaatt |
| 1081 | aatattaatg gaagacatta aatttcctcg aacactctct tcagatgcaa aatcattgct |
| 1141 | ttcagggctc ttgataaagg atccaaataa acgccttggt ggaggaccag atgatgcaaa |
| 1201 | agaaattatg agacacagtt tcttctctgg agtaaactgg caagatgtat atgataaaaa |
| 1261 | gcttgtacct ccttttaaac ctcaagtaac atctgagaca gatactagat attttgatga |
| 1321 | agaatttaca gctcagacta ttacaataac accacctgaa aaatatgatg aggatggtat |
| 1381 | ggactgcatg gacaatgaga ggcggccgca tttccctcaa ttttcctact ctgcaagtgg |
| 1441 | acgagaataa gtctctttca ttctgctact tcactgtcat cttcaattta ttactgaaaa |
| 1501 | tgattcctgg acatcaccag tcctagctct tacacatagc aggggca |

Coding sequence = nucleotide 11-1450

Intramyocardial transplantation of adult stem cells has been proposed as a therapy to repair and regenerate damaged myocardium and to restore cardiac function after acute myocardial infarction (MI). Given their multipotency, low immunogenicity, amenability to ex vivo expansion and genetic modification, autologous bone marrow derived mesenchymal stem cells (MSCs) are suitable for this purpose. Injection of MSCs reduces post-infarction ventricular remodeling and in some cases improves left ventricular function. However prior to the invention, mechanism(s) underlying these therapeutic effects have not been clearly defined. In situ differentiation of the transplanted MSCs into cardiomyocytes and other constituent cardiac cell types has been suggested. Intramyocardial transplantation of MSCs transduced with a retroviral vector overexpressing the survival gene Akt (Akt-MSCs) markedly improves the therapeutic efficacy of MSCs in preventing ventricular remodeling and restoring cardiac function when measured 2 weeks after infarction.

The data described herein shows that therapeutic effects seen with the administration of cells occur in less than 72 hours after infarction. These early dramatic effects cannot be readily attributed to myocardial regeneration or neoangiogenesis, but rather indicate that Akt-MSCs release biologically active factors that exert paracrine actions on the ischemic cardiomyocytes. Under hypoxic stimulation, genetically-modified bone marrow derived MSCs overexpressing the Akt gene release paracrine factors that exert cytoprotective effects on isolated cardiomyocytes. Intramyocardial injection of these substances reduces infarct size, prevents left ventricular dysfunction, and decreases in the number of apoptotic cardiomyocytes in vivo. In addition, no increase in microvessel density was observed in is the treated groups compared to controls 72 hours after the injection of the conditioned medium Thus, a significant portion of the salutary effects of Akt-MSCs transplantation is attributable to protection and functional recovery of ischemic myocardium, instead of, or in addition to, de novo cardiac repair and regeneration. The ability of bone marrow derived MSCs, especially Akt-MSCs, to produce factor(s) capable of protecting cardiomyocytes from cell death has not been previously demonstrated.

Using a large scale microarray gene expression analysis (The Affymetrix GeneChip® Mouse Genome 430 2.0) genes that were consistently and reliably over-expressed or suppressed in murine MSC overexpressing the Akt gene (MSC-Akt) under normoxic or hypoxic conditions were identified. Approximately 650 transcripts were differentially regulated between the MSC-Akt and the wild type MSC under normoxia or hypoxia. The set of 650 transcripts was queried for transcripts encoding for secreted proteins. This analysis revealed 44 transcripts that could account for the cardiac protective role of the MSC cells. The differentially expressed genes identified herein are used to develop protein targeted therapeutic approaches to treating and preventing cardiac disorders. The genes whose expression levels were modulated (i.e., increased or decreased) are summarized in Table 1 are collectively referred to herein as “cytoprotective genes”, “cytoprotective nucleic acids” or “cytoprotective polynucleotides” and the corresponding encoded polypeptides are referred to as “cytoprotective polypeptides” or “cytoprotective proteins.”

Among those of particular interest are the secreted frizzled-related proteins 1-3, pleiotrophin, adrenomedulin, extracellular superoxide dismutase 3 and many angiogenic factors (angiopoietin 4, hepatocyte growth factor, vascular endothelial growth factor A etc). Secreted Frizzled Related Proteins (SFRPs) are soluble molecules capable of modulating Wnt signalling. Sfrp1 and Sfrp 2 have been shown to be upregulated in a model of muscle regeneration. Adrenomedullin (AM) is a hypotensive peptide expressed in cardiac tissue whose plasma levels increase in patients with acute myocardial infarction. Pleiotrophin is a novel growth factor that has been associated with cardiac differentiation and fracture healing and repair.

In conclusion, the data described herein demonstrates, for the first time, that Akt-MSCs secrete cytoprotective factors that exert direct salutary effects on ischemic cardiomyocytes. The therapeutic benefits of Akt-MSCs, at least in the acute phase of infarction, appear to be primarily attributable to diffusible factors from the transplanted cells, that acting in a paracrine fashion reduce infarct size, decrease ventricular remodeling and prevent ventricular dysfunction. Accordingly, these isolated, purified, or recombinant factors represent a novel molecular therapy for prevention of ischemic tissue damage.

Coronary Disorders

Many patients are either at risk for or have suffered from various types of heart failure, including myocardial infarction, symptomatic or unsymptomatic left ventricular dysfunction, or congestive heart failure (CHF). An estimated 4.9 million Americans are now diagnosed with CHF, with 400,000 new cases added annually. This year over 300,000 Americans will die from congestive heart failure. Without therapeutic invention, cardiac muscle does not normally have reparative potential. The ability to augment weakened cardiac muscle as described herein is a major advance in the treatment of cardiomyopathy and heart failure. Despite advances in the medical therapy of heart failure, the mortality due to this disorder remains high, where most patients die within one to five years after diagnosis.

Coronary disorders are categorized into at least two groups. Acute coronary disorders include myocardial infarction, and chronic coronary disorders include chronic coronary ischemia, arteriosclerosis, congestive heart failure, angina, atherosclerosis, and myocardial hypertrophy. Other coronary disorders include stroke, myocardial infarction, dilated cardiomyopathy, restenosis, coronary artery disease, heart failure, arrhythmia, angina, or hypertension.

Acute coronary disorders result in a sudden blockage of the blood supply to the heart which deprives the heart tissue of oxygen and nutrients, resulting in damage and death of the cardiac tissue. In contrast, chronic coronary disorders are characterized by a gradual decrease of oxygen and blood supply to the heart tissue overtime causing progressive damage and the eventual death of cardiac tissue.

Cytoprotective Compounds

A cytoprotective (i.e., cell protective ) compound is a compound that that is capable of inhibiting cell damage such as apoptosis induced or oxidative-stress induced cell death.

Cytoprotective compounds also include compounds that induce cell repair and regeneration. Suitable cytoprotective compounds include, as non-limiting examples, those polypeptides listed in Table 1. A cytoprotective compound is a polypeptide or nucleic acid encoding the polypeptide, the expression of which is increased in MSC-Akt cells under hypoxic conditions as compared to normoxic condition. For example, a cytoprotective polypeptide includes adipsin, adrenomedullin, chemokine (C—C motif) ligand 2, cysteine rich protein 61, lysyl oxidase-like 2, secreted frizzled-related sequence protein 2, serine proteinase inhibitor or vascular endothelial growth factor or fragment thereof. In some aspects the compound is a nucleic acid that increases expression of a nucleic acid that encodes a polypeptide of Table 1 or an agonist of a polypeptide of Table 1.

Alternatively, a cytoprotective compound is a compound that inhibits the expression or activity of a polypeptide of Table 1, the expression of which is decreased under hypoxic condition as compared to normoxic condition. The compound is, for example, an antisense nucleic acid, a short-interfering RNA, or ribozyme specific for a downregulated polypeptide of Table 1, e.g., aggrecanase-2, angiopoietin 4, apolipoprotein D, arginyl aminopeptidase, carboxypeptidase E, chemokine (C—X—C) ligand 12, fibronectin, inhibitin beta, interferon alpha inducible protein, osteoglycin, or superoxide dismutase 3. A decrease in polypeptide expression or activity is defined by a reduction of a biological function of the protein. Protein expression is measured by detecting a MTP transcript or protein.

TABLE 1

| | | | | fold change |
|---|---|---|---|---|
| | | | | MSC-Akt- |
| | | | fold change | Gfp |
| | | | MSC-Akt-Gfp | HYPOXIA |
| | | | NORMOXIA | vs MSC- |
| Affymetrix | | | vs MSC-Gfp | Gfp |
| Probe Set ID | Gene Title | Gene Symbol | NORMOXIA | HYPOXIA |
| 1417867_at | adipsin | And | 3.5 | 4.15 |
| 1447839_x_at | adrenomedullin | Adm | −3.72 | 4.03 |
| 1416077_at | adrenomedullin | Adm | −2.78 | 8.36 |
| 1456404_at | aggrecanase-2 | Adamts5 | −1.22 | −3.08 |
| 1450658_at | aggrecanase-2 | Adamts5 | −1.71 | −2.21 |
| 1422561_at | aggrecanase-2 | Adamts5 | −1.14 | −1.91 |
| 1450325_at | angiopoietin 4 | Agpt4 | 2.43 | 1.6 |
| 1423396_at | angiotensinogen | Agt | −2.48 | −1.51 |
| 1416371_at | apolipoprotein D | Apod | 1.88 | 1.34 |
| 1451243_at | arginyl aminopeptidase (aminopeptidase | Rnpep | −1.34 | −1.86 |
| | B) | | | |
| 1423635_at | bone morphogenetic protein 2 | Bmp2 | −3.82 | −3.19 |
| 1415949_at | carboxypeptidase E | Cpe | −1.33 | −1.6 |
| 1449528_at | c-fos induced growth factor | Figf | −2.27 | −2.14 |
| 1438953_at | c-fos induced growth factor | Figf | −3.02 | −2.09 |
| 1438954_x_at | c-fos induced growth factor | Figf | −3.03 | −1.96 |
| 1420380_at | chemokine (C—C motif) ligand 2 | Ccl2 | −6.73 | 1.01 |
| 1421228_at | chemokine (C—C motif) ligand 7 | Ccl7 | −3.4 | −1.25 |
| 1448823_at | chemokine (C—X—C motif) ligand 12 | Cxcl12 | −1.1 | −1.62 |
| 1416953_at | connective tissue growth factor | Ctgf | −6.01 | −1.57 |
| 1438133_a_at | cysteine rich protein 61 | Cyr61 | −3.93 | −1.18 |
| 1416039_x_at | cysteine rich protein 61 | Cyr61 | −4.61 | 1.04 |
| 1426951_at | cysteine-rich motor neuron 1 | Crim1 | −2.41 | −2 |
| 1437218_at | fibronectin 1 | Fn1 | −1.89 | −1.97 |
| 1416164_at | fibulin 5 | Fbln5 | −1.35 | −1.72 |
| 1451866_a_at | hepatocyte growth factor | Hgf | 2.32 | 2.26 |
| 1418450_at | immunoglobulin superfamily containing | Islr | −1.55 | −2.06 |
| | leucine-rich repeat | | | |
| 1426858_at | inhibin beta-B | Inhbb | −2.27 | −4.34 |
| 1421991_a_at | insulin-like growth factor binding | Igfbp4 | 2.32 | 1.19 |
| | protein 4 | | | |
| 1431591_s_at | interferon, alpha-inducible protein | G1p2 | 4.75 | 2.71 |
| 1419043_a_at | interferon-inducible GTPase | Iigp-pending | 3.97 | 3.15 |
| 1419042_at | interferon-inducible GTPase | Iigp-pending | 4.61 | 3.55 |
| 1448117_at | Kit ligand | Kitl | −1.23 | −1.79 |
| 1426152_a_at | Kit ligand/stem cell factor | Kitl | −1.64 | −2.78 |
| 1418061_at | latent transforming growth factor beta | Ltbp2 | −2.66 | −1.87 |
| | binding protein 2 | | | |
| 1429679_at | leucine rich repeat containing 17 | Lrrc17 | 2.36 | 2.35 |
| 1452436_at | lysyl oxidase-like 2 | Loxl2 | 1.8 | 2.62 |
| 1425985_s_at | mannan-binding lectin serine protease 1 | Masp1 | −1.72 | −1.79 |
| 1423294_at | mesoderm specific transcript | Mest | 2.21 | 1.34 |
| 1419662_at | osteoglycin | Ogn | 2.19 | −1.07 |
| 1449187_at | platelet derived growth factor, alpha | Pdgfa | −2.33 | −1.55 |
| 1448254_at | pleiotrophin | Ptn | 5.21 | 4.48 |
| 1416211_a_at | pleiotrophin | Ptn | 5.68 | 4.79 |
| 1427760_s_at | proliferin | Plf | −3.15 | −2.61 |
| 1416594_at | secreted frizzled-related sequence | Sfrp1 | 2.23 | 1.42 |
| | protein 1 | | | |
| 1448201_at | secreted frizzled-related sequence | Sfrp2 | 10.04 | 11.66 |
| | protein 2 | | | |
| 1448424_at | secreted frizzled-related sequence | Frzb | 3.15 | 3.14 |
| | protein 3 | | | |
| 1435603_at | secreted protein SST3 | SST3 | −1.12 | −1.93 |
| 1429348_at | sema domain, immunoglobulin domain | Sema3c | 2.61 | 1.92 |
| | (Ig), short basic domain, secreted, | | | |
| | (semaphorin) 3C | | | |
| 1419149_at | serine (or cysteine) proteinase inhibitor, | Serpine1 | −6.34 | 10.35 |
| | clade E, member 1 | | | |
| 1417634_at | superoxide dismutase 3, extracellular | Sod3 | 4.31 | 1.61 |
| 1417633_at | superoxide dismutase 3, extracellular | Sod3 | 3.23 | 1.78 |
| 1460302_at | thrombospondin 1 | Thbs1 | 1.03 | −1.84 |
| 1447862_x_at | thrombospondin 2 | Thbs2 | −1.33 | −1.8 |
| 1451959_a_at | vascular endothelial growth factor A | Vegfa | −1.07 | 3.64 |

Therapeutic Methods

The invention provides methods of inhibiting cell or tissue damage and ischemic or reperfusion related injuries. Also included are methods of regenerating injured myocardial tissue. The therapeutic methods include administering to a subject, or contacting a cell or tissue with a composition containing a cytoprotective compound.

Cell/tissue damage is characterized by a loss of one or more cellular functions characteristic of the cell type which can lead to eventual cell death. For example, cell damage to a cardiomyocyte results in the loss contractile function of the cell resulting in a loss of ventricular function of the heart tissue. An ischemic or reperfusion related injury results in tissue necrosis and scar formation.

Injured myocardial tissue is defined for example by necrosis, scarring or yellow softening of the myocardial tissue. Injured myocardial tissue leads to one or more of several mechanical complications of the heart, such as ventricular dysfunction, decrease forward cardiac output, as well as inflammation of the lining around the heart (i.e., pericarditis). Accordingly, regenerating injured myocardial tissue results in histological and functional restoration of the tissue.

The cell is any cell subject to apoptotic or oxidative stress induced cell death. For example, the cell is a cardiac cell such as a cardiomyocyte, a liver cell or a kidney cell. Tissues to be treated include a cardiac tissue, a pulmonary tissue, or a hepatic tissue. For example, the tissue is an muscle tissue such as heart muscle. The tissue has been damaged by disease or deprivation of oxygen.

Cells or tissues are directly contacted with a cytoprotective compound. Alternatively, the cytoprotective compound is administered systemically. The cytoprotective compounds are administered in an amount sufficient to decrease (e.g., inhibit) apoptosis induced or oxidative stress induced cell death as compared to untreated cells or tissues. Cells undergoing apoptosis are identified by detecting cell shrinkage, membrane blebbing, caspase activation, chromatin condensation and fragmentation as is well know in the art. Cell undergoing oxidative stress are identified by detecting an increase production of reactive oxygen species (ROS). A decrease in cell death (i.e., an increase in cell viability) is measured by using standard cell viability measurements such as BrdU incorporation assay and trypan blue exclusion.

The methods are useful to alleviate the symptoms of a variety disorders, such as disorders associated with aberrant cell damage, ischemic disorders, and reperfusion related disorders. For example, the methods are useful in alleviating a symptom of stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, kidney ischemia or myocardial hypertrophy. The disorders are diagnosed and or monitored, typically by a physician using standard methodologies. Alleviation of one or more symptoms of the disorder indicates that the compound confers a clinical benefit, such as a reduction in one or more of the following symptoms: shortness of breath, fluid retention, headaches, dizzy spells, chest pain, left shoulder or arm pain, and ventricular dysfunction

Therapeutic Administration

The invention includes administering to a subject a composition comprising a cytoprotective compound (referred to herein as a “therapeutic compound”).

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other anti-apoptotic agents or therapeutic agents for treating, preventing or alleviating a symptom of a particular cardiac disorder. A therapeutic regimen is carried out by identifying a mammal, e.g., a human patient suffering from (or at risk of developing) an cardiac disorder, using standard methods.

The pharmaceutical compound is administered to such an individual using methods known in the art. Preferably, the compound is administered orally, nasally, topically or parenterally, e.g., subcutaneously, intraperitoneally, intramuscularly, and intravenously. The compound is administered prophylactically, or after the detection of an cardiac event such as a heart attack. The compound is optionally formulated as a component of a cocktail of therapeutic drugs to treat cardiac disorders. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for administration utilizing conventional methods. For example, cytoprotective compounds are formulated in a capsule or a tablet for oral administration. Capsules may contain any standard pharmaceutically acceptable materials such as gelatin or cellulose. Tablets are formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, a conventional filler, and a tableting agent. Other formulations include an ointment, suppository, paste, spray, patch, cream, gel, resorbable sponge, or foam. Such formulations are produced using methods well known in the art.

Cytoprotective compounds are effective upon direct contact of the compound with the affected tissue, e.g. heart muscle. Alternatively, cytoprotective compounds are administered systemically. Additionally, compounds are administered by implanting (either directly into an organ such as the heart or subcutaneously) a solid or resorbable matrix which slowly releases the compound into adjacent and surrounding tissues of the subject. For example, the compound is delivered to the cardiac tissue (i.e., myocardium, pericardium, or endocardium) by direct intracoronary injection through the chest wall or using standard percutaneous catheter based methods under fluoroscopic guidance for direct injection into tissue such as the myocardium or infusion of an inhibitor from a stent or catheter which is inserted into a bodily lumen. Any variety of coronary catheter, or a perfusion catheter, is used to administer the compound. Alternatively, the compound is coated or impregnated on a stent that is placed in a coronary vessel.

The present invention is further illustrated, but not limited, by the following examples.

### EXAMPLE 1

### Manipulation and Evaluation of MSCs

Purification and Retroviral Transduction of Mesenchymal Stem Cells

MSCs were isolated and expanded from the bone marrow of adult Sprague-Dawley male rats (Harlan World Headquarters, Indianapolis) according to the protocol used in our laboratory. The cells were transduced with retrovirus encoding either the reporter gene GFP or both GFP and Akt. Transduction efficiency was assessed by FACS analysis (Becton Dickinson FACS Vantage).

Myocardial Infarction Model

Ligation of the left coronary artery (LCA) was performed using known methods. EKG was performed to confirm the presence of infarction. One hour later 5×10⁶ GFP-MSCs or Akt-MSCs suspended in PBS were injected in 5 different sites at the border zone. An equivalent volume of PBS was injected in control group. In the sham animals the ligature was not tightened and no injection was performed.

Cardiac function measurement

Cardiac function was analyzed 72 hours or 15 days after surgery as well known in the art and described below. A water-filled latex balloon inserted into the LV was connected to a pressure transducer (Stathman P23Db; Gould, Oxnard, Calif.) for continuous measurement of LVSP, heart rate and ±dP/dt; the data were collected with a dedicated on-line system (Mac Lab AD Instruments, Milford, Mass.). After baseline perfusion, solution of 300 nM dobutamine was infused through a side tubing by a digital console drive (Cole-Parmer Instrument Company) at 2% of coronary flow rate for 10-15 min.

Infarct Size Determination

Infarct size at 72 hours was analyzed with planar morphometry in TTC (Sigma Chemicals) stained sections and expressed as ratio of the LV area. Each heart was cut into 5 biventricular sections of similar thickness which were incubated in 1% TTC in PBS (pH 7.4) at 37° C. for 5 minutes and fixed for 12 hours in 10% phosphate-buffered formalin. Both sides of each slice were photographed with a digital camera (Nikon Coolpix 4500) connected to a stereomicroscope (Nikon SMZ 1500). The boundary of the unstained areas (infarcted tissue) was traced in a blinded fashion and quantified with dedicated software (ImageJ from NIH). The sections were then repeatedly washed with PBS, processed and embedded in paraffin for H&E staining and histopathological analysis of the infarct. At 2 weeks the infarct size was determined as wet weight of infarcted and noninfarcted tissue measured on the same hearts used for the function study.

Cardiomyocyte Isolation and Conditioned Medium In Vitro Experiments

ARVCs were isolated were isolated using known methods. Cells were seeded in 12-well plates (Becton Dickinson) precoated with laminin (1 μg/cm2) and left overnight in M199 medium containing a standard cocktail of chemicals. One day later the M199 medium was replaced with α-minimal essential medium α-MEM; from GIBCO) either nonconditioned or conditioned from GFP-MSCs or Akt-MSCs. Hypoxic conditions were created by incubating the cells at 37° C. into an airtight Plexiglas chamber (Billups Rothenberg) with an atmosphere of 5% CO2/95% N2. Oxygen level into the chamber was around 0.5% (oxygen analyzer MAXO₂ from Maxtec). Conditioned medium was generated as follow: 90% confluent GFP or Akt MSCs were fed with serum-free α-MEM and incubated in either a standard normoxic incubator (N-M) or the hypoxic chamber (H-M) for 12 hours. α-MEM served as CTR-M.

Morphological Analysis and Apoptosis Quantification of Isolated Cardiomyocytes

The viability of ARCMs was evaluated on the basis of their morphology: rod shaped cardiomyocytes were considered viable. Six ×100 magnification fields for each of the 3 wells analyzed were blindly evaluated. The number of viable ARVCs grown in normal conditions was considered as baseline. Caspase 3 was determined by using a standard fluorimetric assay kit (SIGMA) in accordance with the manufacturer's recommendations. The results obtained were normalized by protein concentration. TUNEL staining was performed with an in situ apoptosis detection kit (Boehringer Mannheim); ARVCs nuclei were counted after propidium iodide staining.

In Vivo Injection of Concentrated Conditioned Medium

Approximately 5×10⁶ GFP-MSCs or Akt-MSCs were fed with α-MEM containing neither FBS nor antibiotics and left for 12 hours into the hypoxic chamber. After removing cell debris the supernatant was transferred into dedicated ultrafiltration tubes (Amicon Ultra-PL 5 from Milllipore). Following the manufacturer's protocol, the medium was concentrated from 15 ml to 300 μl and then desalted it, retaining all the substances with a molecular weight higher than 5,000 Daltons. CTR-cM was generated the same way without cells in the plates. 600 μl of concentrated medium was injected into one heart as described. Control medium (in the absence of cells) was generated by the same protocol. Cardiac function and infarct size were determined. Apoptotic cardiomyocytes were quantified by TUNEL staining (CardioTACS In Situ Apoptosis kit from Trevigen) and expressed as the proportion of the TUNEL-positive cardiomyocyte nuclei from the total number of cardiomyocytes nuclei. The cardiomyocytes origin was identified by the presence of myofilaments surrounding the nucleus. Endothelial cells were stained with an antibody anti-factor VIII (Zymed) and microvessels, defined as any endothelial cell or group of endothelial cells not surrounded by other cell types, quantified as total number per high-power field.

Statistics

All results are presented as mean plus or minus standard error (SE) and were analyzed with a one-way or two-way ANOVA followed by Bonferroni all pair-wise multiple comparison test. Probability (p) values less than 0.05 were considered statistically significant.

### EXAMPLE 2

### Early Effects of Akt-MSCs Transplantation on Ventricular Function Following Myocardial Infarction

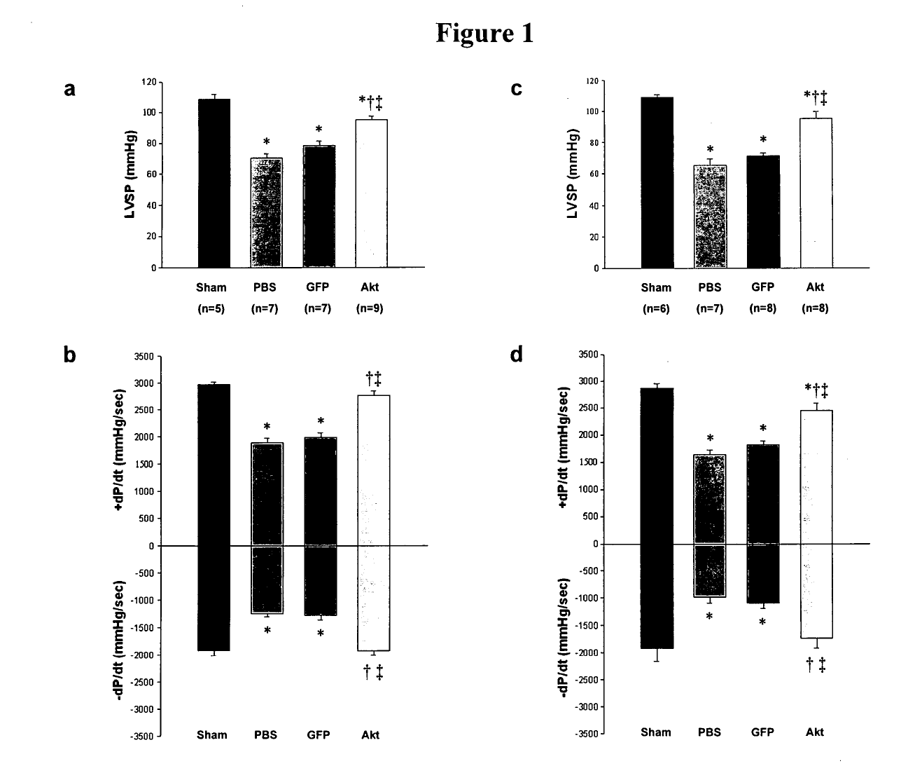

Male rat MSCs were transfected with either a retroviral vector encoding the GFP reporter gene (GFP-MSCs) or with a bicistronic vector expressing both GFP and Akt genes (Akt-MSCs). Fluorescence activated cell sorting (FACS) analysis showed a transduction efficiency of approximately 90% for both viruses. Myocardial infarction and cell transplantation were performed on female adult rats. Before surgery, the animals were randomized into four groups: sham operated animals, control animals that received phosphate buffered solution (PBS) injection, and GFP-MSCs and Akt-MSCs treated animals that were injected with 5×10⁶ cells. Hearts were excised at either 72 hours or 2 weeks post infarction for measurement of contractile performance. Isolated heart preparation allowed the measurement of ventricular function independent of loading conditions and neurohormonal factors. Cells injection, isolated heart experiments and data analysis were performed blinded to the treatment groups. Left ventricular (LV) function measured in isolated perfused isovolumetrically contracting hearts 72 hours and 2 weeks after the infarction are shown in FIG. 1a-d. At 72 hours, the LV systolic pressure (LVSP) of the PBS-injected hearts was 36% lower than sham-operated control hearts (p<0.05) (FIG. 1a). The LVSP of GFP-MSCs treated animals was similar to the PBS group, while injection of Akt-MSCs resulted in significantly higher LVSP than in both PBS and GFP-MSCs groups (p<0.05) (FIG. 1a). In addition the rates of tension development (+dP/dt) and of relaxation (−dP/dt) followed the same pattern (FIG. 1b). Importantly, the differences in LV function achieved at 72 hours after infarction were comparable to those observed at 2 weeks (FIG. 1c,d). Body, heart and LV weights did not differ among the groups at either time point.

### EXAMPLE 3

### The Effect of Akt-MSCs Transplantation on Myocardial Injury

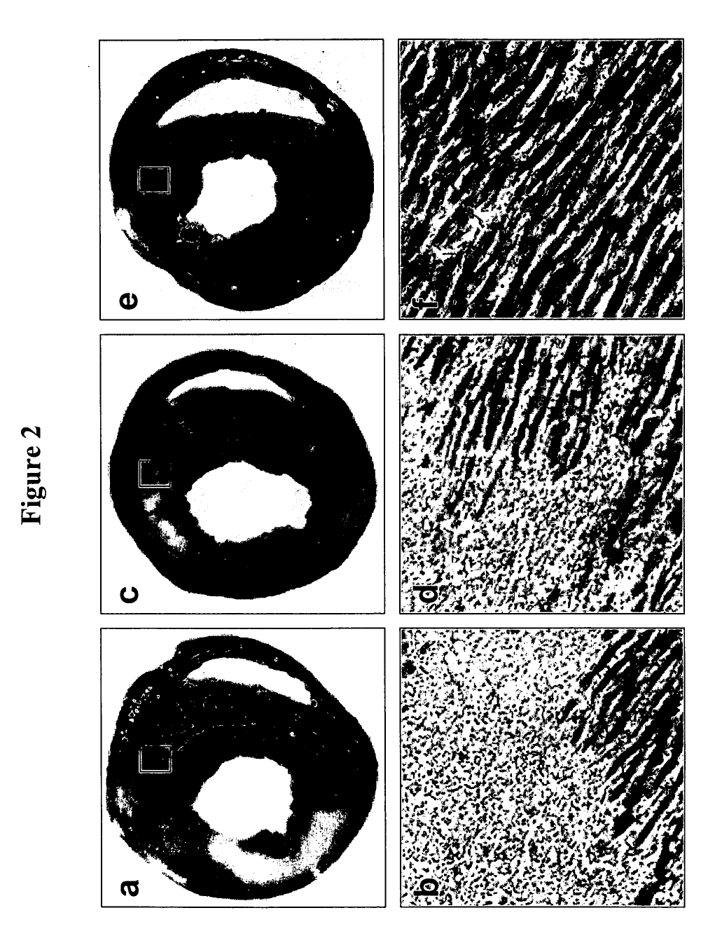

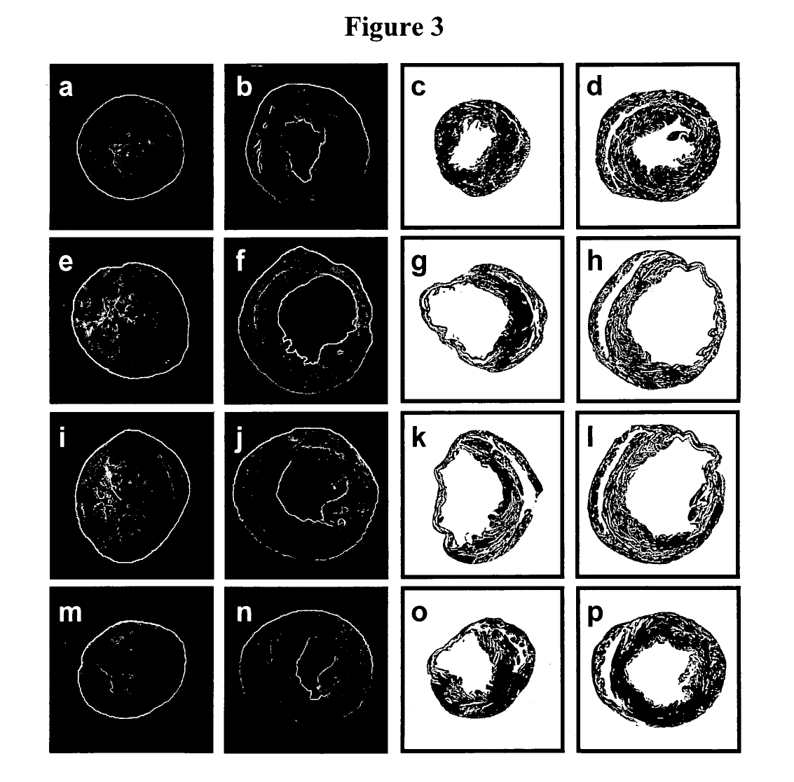

The effect of Akt-MSCs transplantation on myocardial injury at 72 hours after infarction was evaluated by triphenyltetrazolium chloride (TTC) and hematoxylin-eosin (H&E) staining (FIG. 2a-e). Mean infarct size in PBS-control animals was 34±4% of the LV (FIG. 2a). Injection of GFP-MSCs had a modest protective effect, reducing the size of the infarct to 29%±3% (p=NS) (FIG. 2b). In contrast, injection of Akt-MSCs reduced the infarct size to 13%±4 of the LV (p<0.05 vs. PBS and GFP-MSCs groups) (FIG. 2c). These translated into relative reductions in infarct size of 15% and 62% in the GFP and Akt group respectively. Microscopic analysis of H&E-stained sections confirmed the gross morphological observations of myocardial infarction seen in the TTC-stained sections (FIG. 2d-e). The morphological appearance of the LV at 2 weeks after infarction is shown in FIG. 3. No evidence of infarction or LV remodeling was seen in the sham group (FIG. 3a-d). Masson's trichrome staining revealed marked thinning and extensive fibrosis of the infarcted anterolateral wall and significant enlargement of the LV cavity in the PBS-treated control group (FIG. 3e-h). GFP-MSCs transplantation did not significantly reduce the severity of remodeling (FIG. 3i-l). In contrast, Akt-MSCs transplantation dramatically attenuated LV wall thinning and chamber dilatation (FIG. 3m-p).

### EXAMPLE 4

### Akt-MSC Secrete Factors that Protects Adult Cardiomyocytes from Cell Damage Associated with Hypoxia

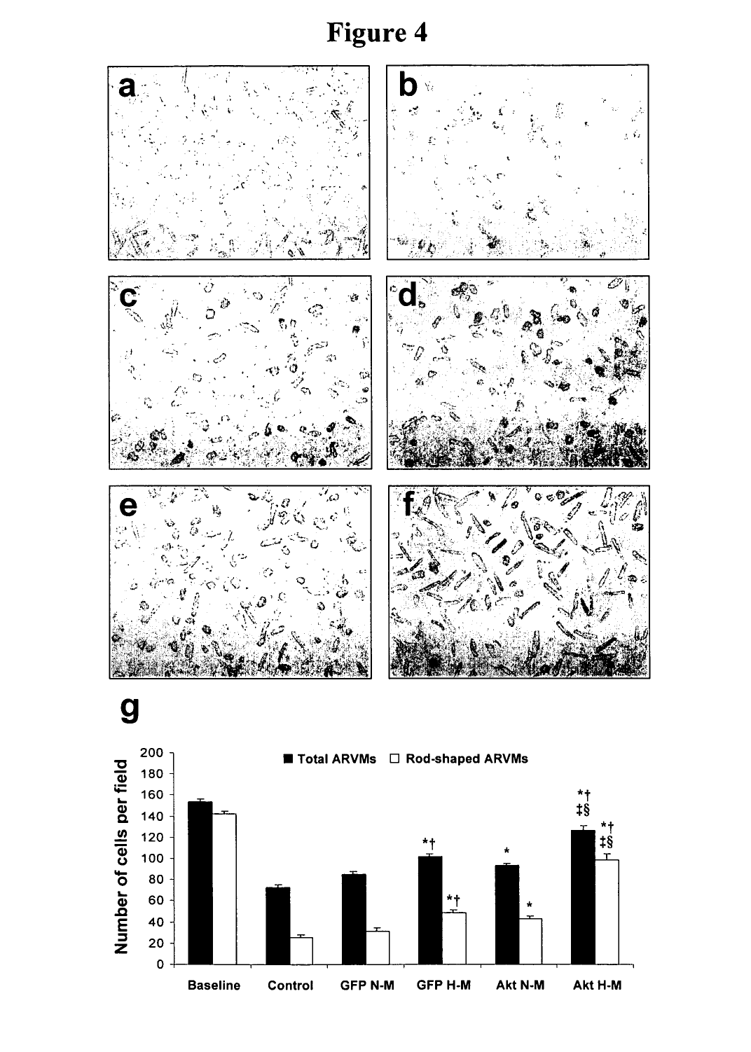

Akt-MSCs release cytoprotective factor(s) that can prevent cardiomyocytes loss. The effects of conditioned medium from cultured MSCs on the viability and function of ARVCs subjected to hypoxia was assessed. To simulate in situ conditions and minimize cell death, the conditioned medium (M) was collected from MSCs after 12 hours of exposure either to normoxia or hypoxia. First the standard growth medium of ARVCs was replaced with control conditioned medium (CTR-M), normoxic conditioned medium (N-M) or hypoxic conditioned medium (H-M) from GFP or Akt-MSCs; the ARVCs were subsequently exposed to hypoxia for 24 hours. ARVCs maintained in basal α-MEM under normoxic conditions for 24 hours were viable and exhibited their typical rod-shaped appearance (FIG. 4a, g). Exposure of ARVCs to 24 hours of hypoxia in CTR-M resulted in a 53% reduction of the total cell number (p<0.05) and 82% decrease of rod-shaped cells (p<0.05) (FIG. 4b, g). The transition of ARVCs from the rod-shaped to the rounded morphology coincides with ultrastructural alterations typical of necrotic and apoptotic cell death. Exposure to GFP-MSCs N-M did not significantly change the number of total or rod-shaped ARVCs (FIG. 4c, g). In contrast Akt-MSCs N-M led to an increase in the total number (+28%; p<0.05) as well as in the prevalence of rod-shaped ARVCs (+67%; p<0.05) compared with CTR-M (FIG. 4d, g). These results demonstrate that under normoxic condition, overexpression of Akt induces the release of cytoprotective factor(s) from the MSCs. The results were even more striking with conditioned medium of MSCs exposed to hypoxia, especially Akt-MSCs. Compared with CTR-M, the GFP-MSCs H-M increased the total cell number by 39% (p<0.05) and the number of rod-shaped ARVCs by 89% (p<0.05) (FIG. 4e, 4g); compared with GFP-MSCs N-M, the increases were 20% and 53% respectively (p<0.05). Under hypoxia, MSCs released factor(s) capable of protecting ARVCs from hypoxia induced damage. Significantly greater protection was achieved with Akt-MSCs H-M (FIG. 4f and 4g): the total number of ARVCs was 73% higher than the number of ARVCs in CTR-M (p<0.05) and the rod-shaped cells were 3.8 fold more numerous (p<0.05). The total number of ARVCs was 36% higher (p<0.05) and the rod-shaped cells were 2.2 fold more (p<0.05) compared to ARVCs in Akt-MSCs N-M, indicating that exposure to hypoxia triggers the production and extracellular release of protective factors from Akt-MSCs. Finally, compared with GFP-MSCs H-M, Akt-MSCs H-M increased the total number by 24% (p<0.05) and the percentage of rod-shaped ARVCs by 2.0 fold (p<0.05).

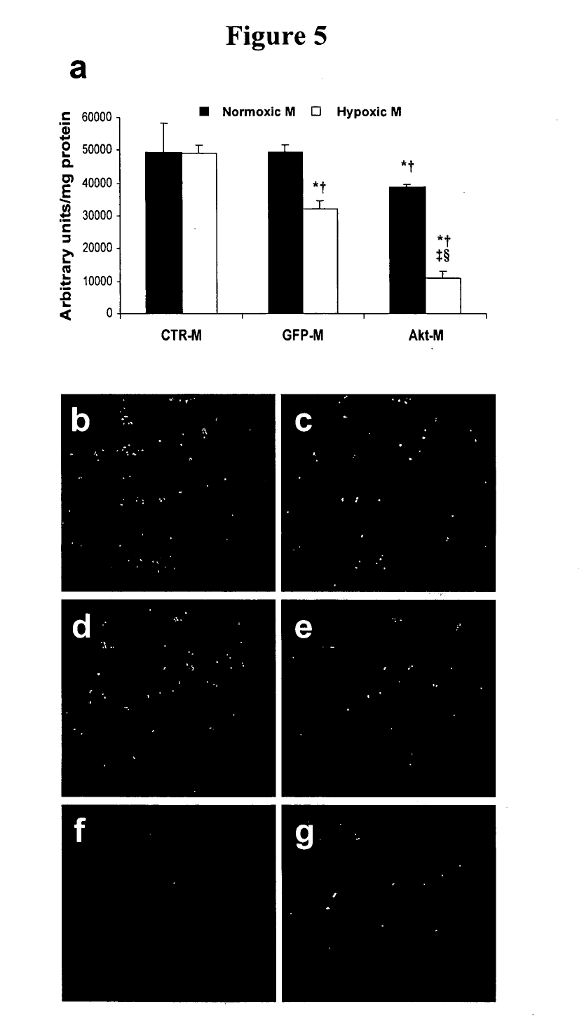

Since apoptosis plays a major role in cell loss in myocardial infarction, experiments were carried out to determine whether the MSCs conditioned medium exerted anti-apoptotic effects. Caspase 3 activity of ARVCs was measured under the same conditions as described above for the analysis of cell number and morphology. It was found that conditioned medium from GFP-MSCs maintained under normoxia had no significant effect on caspase 3 activity. In contrast, conditioned medium from normoxic Akt- MSCs significantly reduced caspase 3 activity by 21% compared with control (p<0.05) (FIG. 5a). Conditioned medium from both GFP-MSCs and Akt-MSCs maintained under hypoxia significantly decreased caspase 3 activity (FIG. 5g) but Akt-MSCs H-M had a more dramatic effect reducing the caspase activity by 66% compared with the GFP H-M (p<0.05) and by 78% compared with CTR-M (p<0.05). The background level, determined for each condition as caspase 3 activity of ARVCs cultured under normoxia, was similar among all the groups and it was subtracted from all the results obtained under hypoxia. Finally it was determined the relative number of apoptotic ARVCs after 24 hours of hypoxia using terminal deoxyribonucleotidyl transferase (TdT)-mediated dUTP nick end-labeling (TUNEL) labeling (FIG. 5b-g). In the presence of Akt-MSCs H-M, there were 62% fewer TUNEL-positive cells (p<0.05) than in presence of CTR-M and 54% fewer than in presence of GFP-MSCs H-M (p<0.05). These results suggest that the factor(s) secreted into the conditioned medium, particularly by Akt-MSCs, were highly protective against apoptosis and that their release is increased under hypoxia.

### EXAMPLE 5

### Compositions (Paracrine Factors) Secreted by Akt-MSCs Induced ARVCs

Spontaneous Contraction Under Prolonged Hypoxia

The behavior of ARVCs in real-time after 48 hours of incubation in the hypoxic chamber was examined. In the presence of CTR-M very few ARVCs were attached to the plates, and almost all of them were rounded-up and did not form clusters. Only 0-5% of the cells showed spontaneous but irregular contractile activity. In the presence of GFP-MSCs H-M, 28% more ARVCs were attached to the plate (p<0.05); some of them maintained their original rod-shape but the majority was rounded-up and the spontaneous contractility was slow and irregular. However the number of ARVCs contracting was higher than in the presence of CTR-M, around 10-15% of the total number of cells left. In striking contrast, significantly more cells were still attached to the plate in the presence of Akt-MSCs H-M (+3.8 folds vs CTR-M; p<0.05). Interestingly, the majority but not all of the ARVCs were rounded-up and tended to cluster. Most importantly 60-65% of them were spontaneously and strongly beating and in some cases the contraction of adjacent cells was synchronized simulating a syncytium.

### EXAMPLE 6

### In Vivo Early Cardiac Protection by Akt-MSCs Paracrine Factors

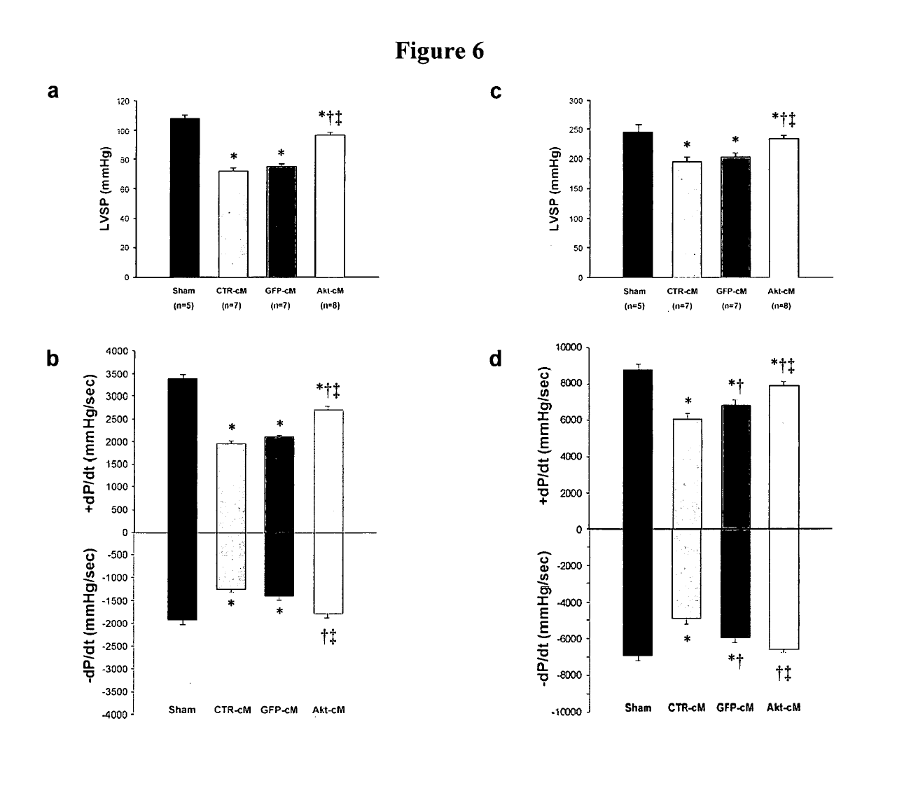

To examine the in vivo relevance of our in vitro findings, the direct effects of the medium containing the putative protective factor(s) on infarct size and ventricular function by injecting the secreted factors into infarcted rat hearts was evaluated. On the basis of the in vitro results and to streamline the design of in vivo experiments, medium only from MSCs exposed to hypoxia was used. Concentrated medium (cM) was injected into 5 different sites in the heart at the infarct border zone 30 minutes after LCA occlusion. Hearts were isolated 72 hours later to define contractile performance (FIG. 6a,b). Compared with control concentrated medium (CTR-cM), injection of concentrated medium from GFP-MSCs (GFP-cM) resulted in small but not significant improvements of LVSP (FIG. 6a), +dP/dt and −dP/dt (FIG. 6b). In contrast, injection of conditioned medium from Akt-MSCs (Akt-cM) significantly improved LVSP, +dP/dt and −dP/dt, compared with hearts treated with either CTR-cM or GFP-cM (FIG. 6a, b). The inotropic response of ventricular function to dobutamine stimulation (FIG. 6c, d) was also analyzed. The Akt-cM treated hearts exhibited significantly enhanced inotropic response compared with the other groups (FIG. 6c, d). In these same hearts, the infarct size was measured by TTC staining after studying their ventricular function. In CTR-cM control animals the infarct size was 33±5% of LV area. Injection of GFP-cM reduced the infarct to 29±4% (p=NS) of the LV. Injection of concentrated conditioned medium from Akt-MSCs reduced the infarct size to 15±4% (p<0.05). To confirm the anti-apoptotic action of the Akt-cM in vivo, TUNEL staining was performed and quantified the positive cells at the border zone of the infarction. The cardiomyocyte apoptotic index was reduced in the GFP-cM group as compared with CTR-cM group (CTR-cM: 10.1±1.1% vs GFP-cM: 8.6±1.0%; p=NS). In contrast, a striking anti-apoptotic effect was observed after injection of Akt-cM, yielding a 69% reduction of TUNEL positive cardiomyocytes compared with CTR-cM (CTR-cM: 10.1±1.1% vs AKT-CM: 3.2±1.0%; p<0.05) and a 63% reduction compared with GFP-cM group (GFP-cM: 8.6±1.0%; Akt-cM: 3.2±1.0%; p<0.05). Finally, capillary density was quantified to examine the possible contribution of neoangiogenesis. The number of micro vessels per high power field was not statistically different among the groups (CTR-cM: 161±19; GFP-cM: 157±20; AKT-cM: 179±23; p=NS).

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. A method of restoring a biological function of a bodily tissue, comprising contacting an injured or diseased tissue with a composition comprising a paracrine factor of a mesenchymal stem cell (MSC), wherein said function is increased in the presence of said factor compared to in the absence of said factor.

2. The method of claim 1, wherein said MSC comprising a recombinant Akt gene.

3. The method of claim 1, wherein said composition comprises a mixture of at least two paracrine factors.

4. The method of claim 1, wherein said composition comprises a cell culture supernatant of said MSC.

5. The method of claim 1, wherein said function is selected from the group comprising cell repair, cell regeneration, angiogenesis, inotropy, and tissue morphogenesis.

6. The method of claim 1, wherein said factor is secreted frizzled-related sequence protein 2.

7. The method of claim 1, wherein said tissue is heart tissue.

8. The method of claim 1, wherein said tissue is selected from the group consisting of heart, brain, kidney, liver, pancreas, lung, stomach, intestine, prostate, cervix, and breast.

9. The method of claim 1, further comprising contacting said injured or diseased tissue with a cell transplant.

10. A method of producing a therapeutic agent, comprising providing a MSC, contacting said MSC with a stress condition or substance, and retrieving an extracellular composition from said MSC, wherein said composition comprises a therapeutic agent.

11. The method of claim 10, wherein said MSC comprises a recombinant Akt gene.

12. The method of claim 10, wherein said stress condition is hypoxia.

13. The method of claim 10, wherein said stress substance is a cytotoxic compound.

14. The method of claim 13, wherein said compound is a carcinogen.

15. A method of identifying a therapeutic agent, comprising contacting a MSC with a stress condition or substance and identifying differential expression of a gene product, wherein said differentially-expressed gene product comprises a therapeutic agent.

16. The method of claim 15, wherein said MSC comprises a recombinant Akt gene.

17. The method of claim 15, wherein said differentially-expressed gene product is increased in the presence of said condition or substance compared to in the absence of said condition or substance.

18. The method of claim 15, wherein said differentially-expressed gene product is decreased in the presence of said condition or substance compared to in the absence of said condition or substance.
